(19) United States
(12) Patent Application Publication
(10) Pub. No.: US 2017/0184492 A1
(43) Pub. Date: Jun. 29, 2017

(54) GAS ANALYZER SYSTEM

(71) Applicant: COMDEK INDUSTRIAL CORPORATION, TAIPEI (TW)

(72) Inventors: Hsiang-Ann Hong, New Taipei City (TW); Chang-Yu Tsai, Taipei City (TW); Chien-Pang Kuo, New Taipei City (TW); Yi-Sung Kuo, New Taipei City (TW)

(21) Appl. No.: 14/979,465

(22) Filed: Dec. 27, 2015

(51) Int. Cl. G01N 21/25; G01N 21/31; A61B 5/08; G01N 33/00

(52) U.S. Cl. CPC G01N 21/255; G01N 33/004; G01N 21/31; A61B 5/08; G01N 2201/08

(57) ABSTRACT

According to one embodiment, the gas analyzer system comprises a gas adaptor, a light guide tube and a measure case. The gas adaptor is used for guiding a gas to be detected, wherein the gas is flowed in the gas adaptor along a flow direction. The light guide tube embedded in the gas adaptor, wherein the light guide tube has a light source end and a sensor end, the light guide tube has at least one aperture for the gas entering the light guide tube, and a normal vector of a cross surface defined by the aperture has an angle with respect to the gas inhale direction, wherein the angle is not 180 degrees. The measuring case for accommodating the gas adaptor comprises a light source corresponding to the light source end and a sensor corresponding to the sensor end of the light guide tube.

### BACKGROUND OF THE INVENTION

1. Field of the Invention

The present invention is related to a gas analyzer system, and more particularly, to a respiratory gases analyzer system that is used to detect carbon dioxide concentration of a patient.

2. Description of the Prior Art

Gas sensors are commonly employed in industrial and consumer applications to measure analytes in the gaseous state. Many gas sensors rely on the absorption characteristics of the target analyze when illuminated with radiation and comprise a radiation source, a detector capable of detecting radiation emitted by the radiation source, and a chamber for receiving the target gaseous analyte. Analyte gas within the chamber absorbs radiation of specific wavelengths or ranges of wavelengths and the attenuation of the radiation detected by the detector gives an indication of the concentration of the target analyte within the chamber.

In clinical use, various types of sensors that are configured to communicate with the airway of a patient to monitor substances such as gases or vapors in the respiration of the patient are known in the art. Molecular oxygen, carbon dioxide and anesthetic agents, including nitrous oxide, are among the types of substances that may be detected with known sensors.

### SUMMARY OF THE INVENTION

The present invention therefore provides a gas analyzer system, and more particularly, to a respiratory gases analyzer system that is used to detect carbon dioxide concentration of a patient.

According to one embodiment, the gas analyzer system provided in the present invention comprises a gas adaptor, a light guide tube and a measure case. The gas adaptor is used for guiding a gas to be detected, wherein the gas is flowed in the gas adaptor along a flow direction. The light guide tube is embedded in the gas adaptor, wherein the light guide tube has a light source end and a sensor end, the light guide tube has at least one aperture for the gas entering the light guide tube, and a normal vector of a cross surface defined by the aperture has an angle with respect to the gas inhale direction, wherein the angle is not 180 degrees. The measure case is used for accommodating the gas adaptor, wherein the measure case comprises a light source corresponding to the light source end and a sensor corresponding to the sensor end of the light guide tube.

In our gas analyzer system, by using a novel structure of light guide tube, the elongated pathway can increase the sensitivity of the device. In addition, one preferred embodiment of the light source and the sensor are at the same side/plane of the measure case, making it easy to install. Moreover, the aperture on the light guide tube does not face the gas flow direction, thus unwanted impurities would not enter the light guide tube.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 shows a schematic diagram of the gas analyzer system according to one embodiment of the present invention.

FIG. 2 shows one schematic diagram of the measure case according to one embodiment of the present invention.

FIG. 3 shows one schematic diagram of the gas adaptor according to one embodiment of the present invention.

FIG. 4 shows a cross sectional view of the gas adaptor and the light guide tube.

FIG. 5 shows a schematic diagram of the aperture according to one embodiment of the present invention.

FIG. 6 shows a schematic diagram of the inhale direction and the apertures.

FIG. 7 shows a schematic diagram of the gas adaptor according to one embodiment of the present invention.

FIG. 8 shows schematic diagram of the gas adaptor according to an embodiment of the present invention.

FIG. 9 shows a schematic diagram of the use of the gas analyzer system according to one embodiment of the present invention.

### DETAILED DESCRIPTION

To provide a better understanding of the presented invention, preferred embodiments will be described in detail. The preferred embodiments of the present invention are illustrated in the accompanying drawings with numbered elements.

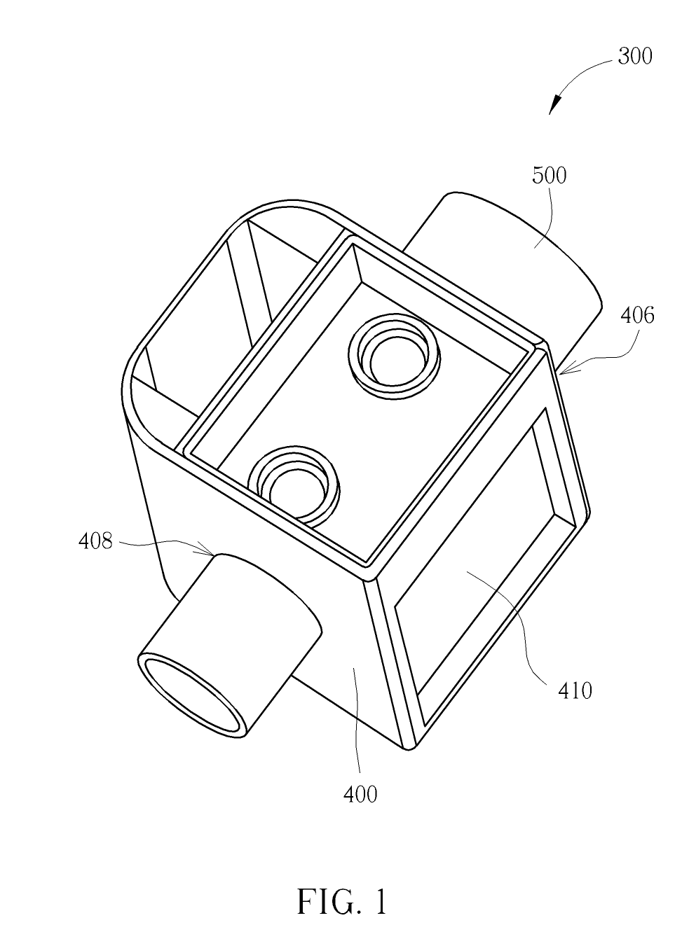

The present invention is directed to a gas analyzer system that is used to analyze a composition of a gas. In particular, the gas analyzer system set forth in the present invention is used to monitor and measure a carbon dioxide (CO₂) concentration of a patient. Please see FIG. 1, which shows a schematic diagram of the gas analyzer system according to one embodiment of the present invention. As shown in FIG. 1, the gas analyzer system 300 includes a measure case 400 and a gas adaptor 500 embedded into the measure case 400. The gas adaptor 500 is used to provide a route for a gas to pass through and the light detection system therein (not shown in FIG. 1) can therefore analyze the gas. A monitor 410 can display the detecting results in real time. The measure case 400 provides a support for the gas adaptor 500 and can be further coupled to other components with different functions, depending on the design of the product. For example, the measure case 400 can further couple to any motif to enable its portable function, such as any wireless technology, so as to connect to other device.

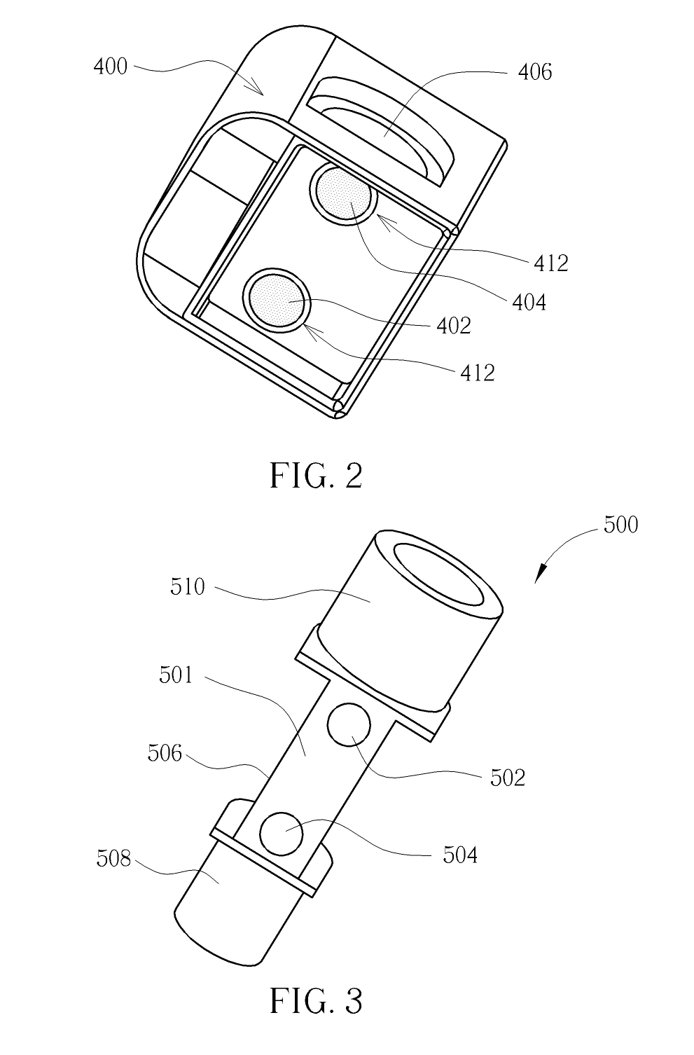

Please refer to FIG. 2, which shows one schematic diagram of the measure case according to one embodiment of the present invention. The measure case 400 can be of any shape that can accommodate and support the gas adaptor 500. As shown in FIG. 2, the measure case 400 is a box like structure, having two opposite openings 406 and 408 (shown in FIG. 1) for holding and containing the gas adaptor 500. The measure case 400 further includes a light source 402 and a sensor 404, for emitting a light energy and detecting the light energy respectively. The light source 402 can be any type of light with different wavelength, such as visible light or infrared rays and is not limited thereto. In the present embodiment, the light source 402 and the sensor 404 are disposed at the same side/plane of the measure case 400 in order to respectively coincide with two ends of the light guide tube (not shown, will be described below). The light source 402 and the sensor 404 can be positioned inside the measure case 400 or, they can be lodged into the openings 412 of the measure case 400. Alternative, they can even be placed outside the measure case 400 and connected to the openings 412 through manners that would not reduce their light energy, such as optical fiber. By doing this, different light source 402 can be changed easily in order to measure different gas.

Please refer to FIG. 3, which shows one schematic diagram of the gas adaptor according to one embodiment of the present invention. As shown in FIG. 3, the gas adaptor 500 has a front portion 508, a middle portion 506 and an end portion 510. When embedded into the measure case 400, the front portion 508 and the end portion 510 are located outside the measure case 400 to provide a gas input and a gas output (please see back to FIG. 1). In one preferred embodiment, both the front portion 508 and the end portion 510 are co-axially hollow cylindrical. Preferably, the diameters of front portion 508 and end portion 510 are in accordance with different regulated sizes, so they can more easily connect other tubes and ensure the gas inhale direction. However, they can have different shapes according to different requirements. The middle portion 506 can be any suitable shape depending on the measure case 400. Preferably, the middle portion 506 includes a planar surface 501, and when the gas adaptor 500 is assembled into the measure case 400, the planar surface 501 would correspond to the side where the light source 402 and the sensor 404 are located.

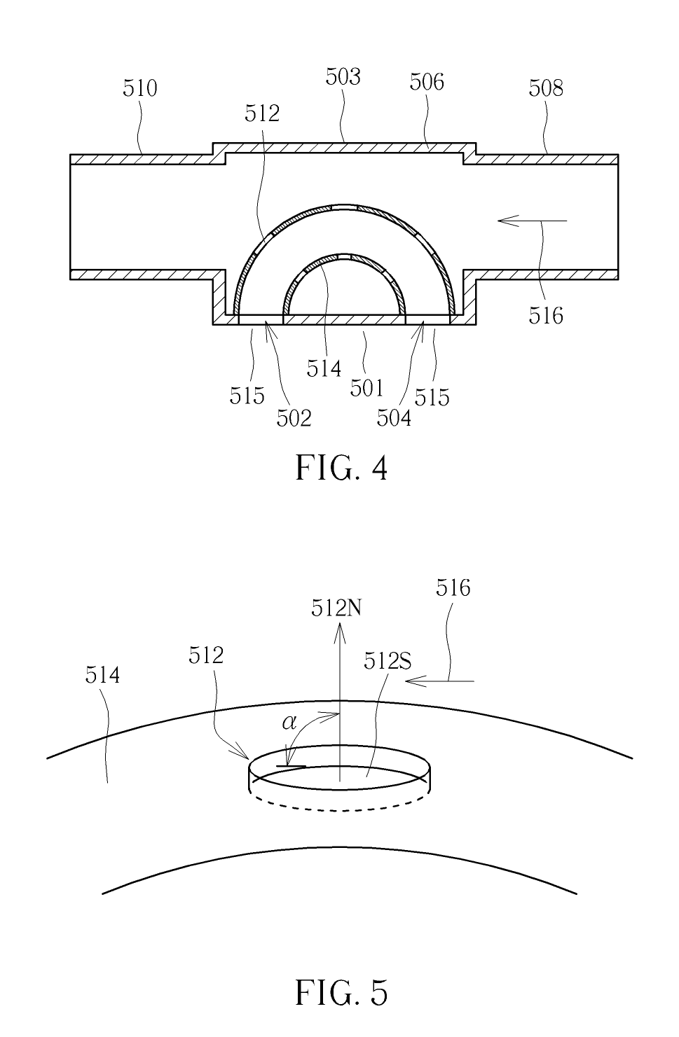

Please refer to FIG. 4, which shows a cross sectional view of the gas adaptor and the light guide tube. The gas adaptor 500 has a light guide tube 514 disposed in the middle portion 506. The light guide tube 514 is used to allow light to pass through and is preferably made of or coated by a material that does not absorb light energy. In one embodiment, the material thereof is a metal such as copper. In another embodiment, the light guide tube 514 can be an optical fiber. As shown in FIG. 4, the light guide tube 514 has a U shape in its cross-section, with one end (a light source end 502) positioned on one side (the planar surface 501 for example) and the other end (sensor end 504) positioned on the same side (the planar surface 501 for example). The light source end 502 would coincide with the light source 402 in the measure case 400 while the sensor end 504 would coincide with the sensor 404 in the measure case 400. Accordingly, light emitted by the light source 402 can pass through the light guide tube 514 and detected by the sensor 404. Since the light guide tube 514 has a U shape, the light pathway is elongated comparing to conventional arts, thereby improving the sensitivity of the device. In one optional embodiment, two windows 515 on the gas adaptor 500 can be disposed between the light source end 502 and the light source 402, the sensor end 504 and the sensor 404. In this embodiment, the window 515 is preferably made of a material that absorb very low amount of light energy. In another embodiment, two ends of the light guide tube 514, the light source end 502 and the sensor end 504, can be directly lodged into the gas adaptor 500 for directly contacting the light source 402 and the sensor 404.

A plurality of apertures 512 are formed on the light guide tube 514, allowing the gas to enter the light guide tube 514. After the light energy absorbed by the gas in the light guide tube 514, the remaining reduced light energy will be captured by the sensor 404. A computing unit (not shown) in the measure case 400 will then detect the remaining reduced amount of light energy, and an analyzed result (a concentration of CO₂ for example) will be calculated by algorithm. A monitor 410 (shown in FIG. 1) can display the detecting results in real time.

Please refer to FIG. 5, which shows a schematic diagram of the aperture according to one embodiment of the present invention. As shown in FIG. 5, a diameter of each aperture 512 is substantially between 1 mm and 3 mm, but is not limited thereto. The aperture 512 of the present invention can have any shapes, preferably a circle. It is one salient feature of the present invention that the apertures 512 on the light guide tube 514 would not “face” the gas inhale direction 516. Detail speaking, a gas inhale direction 516 is not perpendicular to a cross surface 512S defined by the aperture 512. The gas inhale direction 516 and a normal vector 512N of the cross surface 512S has an angle α, wherein the angle α is not 180 degrees. Preferably, the angle α is substantially between 0 degree and 135 degrees, more preferable, between 0 degree and 120 degrees, and most preferably, the angle α is between 0 degree and 90 degrees (The cross surface 512S of the present invention is defined as the topmost surface of the aperture 512 relative to the light guide 514 and the normal vector 512N thereof points to a direction away from the light guide 514). In this manner, unwanted impurities, such as the secretions of patients, would not easily enter the light guide tube 514 through the aperture 512 to obstruct the way of light, so the sensitivity of the device can be improved.

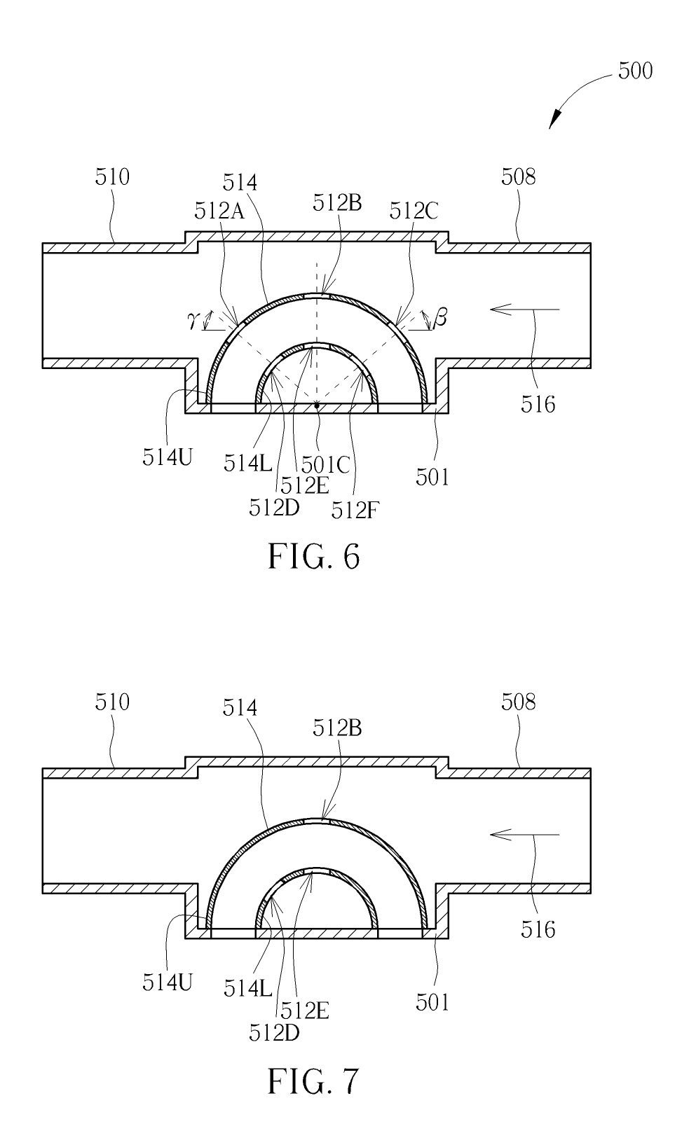

Please refer to FIG. 6, which shows a schematic diagram of the inhale direction and the apertures. As shown in FIG. 6, six apertures 512A, 512B, 512C, 512D, 512E, 512F are dispersed evenly on the light guide tube 514, with three apertures 512D, 512E, 512F on a lower surface 514L which is near the planar surface 501, and three apertures 512A, 512B, 512C on an upper surface 514U which is opposite to the planar surface 501. The aperture 512A corresponds to the aperture 512D, the aperture 512B corresponds to the aperture 512E and the aperture 512C corresponds to the aperture 512F. Preferably, a normal line of the aperture 512A and a normal line of the aperture 512D are co-axial and directed to a center 501C of a portion on the planar surface 501 that is straddled by the U shaped light guide tube 514. Similarly, a normal line of the aperture 512B and a normal line of the aperture 512E are co-axial and directed to the center 501C; a normal line of the aperture 512C and a normal line of the aperture 512F are co-axial and directed to a center 501C. In one embodiment, an angle β between the normal lines of the apertures 512C, 512F is substantially between 45 degrees and 90 degrees, and an angle γ between the normal lines of the apertures 512A, 512D is substantially between 0 degree and 90 degrees, but is not limited thereto.

When a gas flow into the gas adaptor 500 along the gas inhale direction 516, it will enter the light guide tube 514 via the apertures 512C, 512D, 512E, 512F and leave by the apertures 512A, 512B. The apertures 512C, 512D, 512E, 512F therefore serve as inhale holes and the apertures 512A, 512B serve as exhale holes. It is noted that the gas inhale direction 516 is preferably decided by the inner cylinder shape of the front portion 508. In another embodiment, a filter (not shown) can be installed to the front portion 508 thereto further confine the gas inhale direction 516. Said filter can have other functions such as absorption the water vapor of the detected gas, thereto avoid disturbance from the water vapor. By setting the apertures 512 in the positions mentioned above, a greater volume of gas can enter the gas adaptor 514, thereto increase the sensitivity of the device.

Please refer to FIG. 7, which shows a schematic diagram of the gas adaptor according to one embodiment of the present invention. As shown in FIG. 7, only three apertures 512B, 512D 512E are provided on the light guide tube 514. In this embodiment, the apertures 512D, 512E are disposed on the lower surface 514L in which the aperture 512E is disposed at the middle and the aperture 512D is disposed near the end portion 510. The aperture 512B is disposed on the upper surface 514U and is disposed at the middle, corresponding to the aperture 512E. In this embodiment, the apertures 512D, 512E serve as inhale holes and the aperture 512B servers as an exhale holes. In this embodiment, since there is no aperture 512C, which is closest to the front portion 508, the possibility of unwanted impurities entering the light tube guide tube 514 can further be decreased.

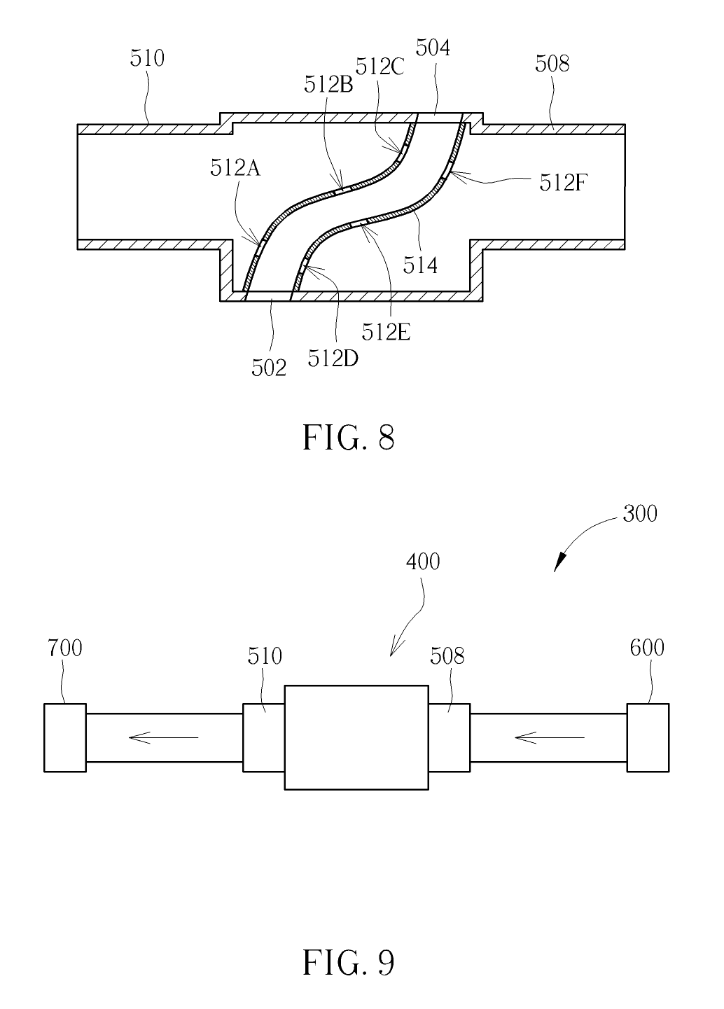

Please refer to FIG. 8, which shows a schematic diagram of the gas adaptor according to one embodiment of the present invention. FIG. 8 shows an alternative embodiment of the light guide tube 514. In the present embodiment, the light guide tube 514 has a serpent structure wherein the light source end 502 and the sensor end 504 are located at two opposite sides of the gas adaptor 500. It is noted that in the present embodiment and the previous embodiment, the light source end 502 and the sensor end 504 of the light guide tube 514 are not aligned with each other, meaning that the projects of the two ends on one surface does not overlap. The apertures 512 are dispersed evenly on the light guide tube 514, with three apertures 512A, 512B, 512C near the end portion 510 and three apertures 512D, 512E, 512F near the front portion 508, wherein the gas inhale direction 516 and a normal vector 512N of the cross surface 512S of each aperture 512 has an angle α which is not 180 degrees. In another embodiment, the aperture 512F can be omitted. It is understood that since the position of the light source end 502 and the sensor end 504 are changed with comparing to the previous embodiment, the light source 402 and the sensor 404 on the measure case 400 are correspondingly changed. Detailed figure of the measure case 400 is omitted.

Please refer to FIG. 9, which shows a schematic diagram of the use of the gas analyzer system according to one embodiment of the present invention. As shown in FIG. 9, the front portion 508 of the gas analyzer system 300 is connected to a gas source 600 and the end portion 510 is connected to an exhale unit 700. In a clinical use, the gas source 600 is a breathing tube installed on a patient, and the exhale unit 700 can be a resuscitator (bag valve mask), an anaesthesia apparatus or atmosphere environment and are not limited thereto. It is understood that depending on different applications, the gas analyzer system 300 can connect to other devices.

In summary, the present invention provides a gas analyzer system. By using a novel structure of light guide tube, the elongated pathway can increase the sensitivity of the device. In addition, one preferred embodiment of the light source and the sensor are at the same side/plane of the measure case, making it easy to install. Moreover, the aperture in the light guide tube does not face the gas inhale direction, thus unwanted impurities would not enter the light guide tube.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A gas analyzer system, comprising:
a gas adaptor for guiding a gas to be detected, wherein the gas is flowed into the gas adaptor along a gas inhale direction;
a light guide tube embedded in the gas adaptor, wherein the light guide tube has a light source end and a sensor end, the light source guide tube has at least one aperture for the gas entering the light guide tube, and a normal vector of a cross surface defined by the aperture has an angle with respect to the gas inhale direction, wherein the angle is not 180 degrees; and
a measure case for accommodating the gas adaptor, wherein the measure case comprises a light source corresponding the light source end and a sensor corresponding the sensor end of the light guide tube.

2. The gas analyzer system according to claim 1, wherein the angle is substantially between 0 degree and 135 degrees.

3. The gas analyzer system according to claim 1, wherein the angle is substantially between 0 degree and 120 degrees.

4. The gas analyzer system according to claim 1, wherein the gas adaptor has a front portion, a middle portion and an end portion.

5. The gas analyzer system according to claim 4, wherein the light guide tube is disposed in the middle portion.

6. The gas analyzer system according to claim 4, wherein the front portion and the end portion is outside the measure case.

7. The gas analyzer system according to claim 1, wherein the light guide tube has a U shape having a lower surface near the light source and an upper surface opposite to the lower portion in its cross section.

8. The gas analyzer system according to claim 7, wherein the light guide tube has a plurality of apertures disposed evenly on the light guide tube.

9. The gas analyzer system according to claim 8, wherein at least two apertures are disposed at the middle of the light guide tube, one on the lower surface and the other on the upper surface.

10. The gas analyzer system according to claim 9, further comprising one aperture disposed on the lower surface and adjacent to the end portion.

11. The gas analyzer system according to claim 10, there are only three apertures on the light guide tube.

12. The gas analyzer system according to claim 7, wherein there are six apertures on the light guide tube with three apertures being disposed evenly on the upper surface and three apertures being disposed evenly on the lower surface of the light guide tube.

13. The gas analyzer system according to claim 1, wherein the light guide tube has a serpent structure.

14. The gas analyzer system according to claim 1, wherein the light source end and a sensor end of the light guide tube are not aligned with each other.

15. The gas analyzer system according to claim 1, wherein the light source end and the sensor end of the light guide tube are disposed at the same side of the measuring case.

16. The gas analyzer system according to claim 1, wherein the light source end and the sensor end of the light guide tube are disposed at two opposite surfaces of the measuring case.

17. The gas analyzer system according to claim 1, wherein a diameter of aperture is substantially between 1 mm and 3 mm.

18. The gas analyzer system according to claim 1, wherein the light guide tube is made of or coated by a metal.

19. The gas analyzer system according to claim 18, wherein the metal is copper.

20. The gas analyzer system according to claim 1, wherein the gas to be detected is carbon dioxide.
